(19) United States
(12) Patent Application Publication
(10) Pub. No.: US 2008/0050451 A1
(43) Pub. Date: Feb. 28, 2008

(54) Methods for assessing dehydration and shock, assays and kits for the methods

(76) Inventor: Helen C. Mabry, Santa Monica, CA (US)

Correspondence Address: WEAN KHING WONG;ATTORNEY AT LAW, PMB 210, 1441 Huntington Drive, South Pasadena, CA, 91030, US

(21) Appl. No.: 11/820,413

(22) Filed: Jun. 18, 2007

Related U.S. Application Data
(60) Provisional application No. 60/814,472, filed on Jun. 16, 2006.

(51) Int. Cl. C12Q 1/40; A61K 33/00; A61P 3/12; C12M 1/34

(52) U.S. Cl. 424/600; 435/22; 435/287.1

(57) ABSTRACT

The present invention is in the medical and consumer arenas. The invention presents the use of a salivary amylase assay to diagnose, quantify, and monitor the hydration status, the onset and progress of shock in an animal that produces salivary amylase, such as a human. The assay tests the saliva sample from the animal. Test kits and assays for such use are also presented.

This application is a continuation-in-part of U.S. provisional patent application, Ser. No. 60/814,472, filed on Jun. 16, 2006. The entire content of the parent application is hereby expressly incorporated by reference.

### TECHNICAL FIELD OF THE INVENTION

The present invention is in the medical and consumer arenas. The invention presents the use of a salivary amylase assay to diagnose, quantify, and monitor the hydration status, the onset and progress of shock in an animal, such as a human. Preferably, the assay tests the saliva sample from the animal. The assays and assay kits for such use are also presented.

### BACKGROUND OF THE INVENTION

Shock is the condition whereby the body is not receiving enough oxygen delivery to the tissues. Shock can be due to blood loss, dehydration, or loss of blood pressure. Shock can also be caused by heart problems, insufficient blood volume, allergic reaction, infections, and damage to the nervous system. Shock is life threatening, because if left unchecked, it will cause organ failure and result in death. Unfortunately, shock can worsen and death can occur very rapidly without immediate medical treatment. Therefore, it is imperative that medical professionals be able to quickly diagnose that the patient is suffering from shock.

Unfortunately, the current art is lacking in a quick, easy and accurate (effective and efficient) means for diagnosing shock, particularly in its early stage. Clearly, the lack of a quick, easy and accurate test and the difficulty in determining whether a patient is in shock, are exacerbated in an emergency, such as in an emergency room, especially because the patient may have other critical conditions or injuries, and the speed at which shock can escalate into a life-threatening condition and death.5

Currently, the diagnosis of shock depends on observing the following signs or symptoms of shock: (1) low blood pressure; (2) elevated heart rate; (3) low or no urine output due to inadequate blood flow to the kidneys; (4) pale, cool, clammy skin; and (5) reduced mental function, impaired speech, confusion or coma due to low blood flow into the brain. However, these diagnoses are not quick, easy and/or accurate. There is currently no efficient and effective way to measure the early states of shock.

A goal of the present invention is to provide a quick, easy, and effective method to detect and monitor dehydration and shock, so that appropriate actions may be taken to reverse their course.

### SUMMARY OF THE INVENTION

One aspect of the invention presents the use of an animal's saliva sample to assay for salivary amylase in the saliva, to assess and quantify the status and extent of hydration (e.g., hydration by fluid such as water and blood) and end organ perfusion of the animal, in order to diagnose, quantify, and/or monitor for resolution, the hydration status (e.g., due to blood loss, or fluid loss such as through sweat loss, fluid loss through breathing/exhalation, or fluid loss due to diseases such as diarrhea), end organ perfusion, onset of shock, the extent of shock if it has set in, and/or to monitor the progress of one or more of the foregoing in order to assess the efficacy of the treatments applied to the animal and to determine whether some other factors are at play. For example, a patient is monitored while he is being treated for shock by being infused with blood and fluid (such as balanced salt solutions) and/or administered with drugs (e.g., drugs to induce vasoconstriction), in order to determine whether the treatments are effective; if not, then some other diagnoses must be explored and other treatments applied.

Another aspect of the invention presents the use of saliva samples to assay for salivary amylase in the saliva of a user, to assess and quantify the status and extent of hydration and end organ perfusion of the user as he physically exerts himself, as he is exposed to the environment, or as he is generally involved in an activity or is in an environment which may expose him to dehydration. By indicating the status and extent of hydration, the invention enables the user to take appropriate preventive or remedial measures, such as periodically drinking fluid, in order to prevent dehydration or shock. A third party may also use the invention to periodically assess and monitor the user, so that the third party may alert the user to employ or provide the user with appropriate preventive or remedial measures.

Another aspect of the invention presents salivary amylase assays and test kits for assaying the saliva sample of an animal. Also presented is the use of such assays and test kits to assess and quantify the status, extent and progress of hydration, end organ perfusion, onset of shock, or progress of and resolution of shock in the animal. The test kits preferably also contain supplies to combat hydration and/or shock.

### BRIEF DESCRIPTION OF THE DRAWINGS

FIG. 1 presents a non-limiting example of a test strip of the present invention. The test strip is rectangular and contains seven reaction zones. Each reaction zone is circular in shape and illustrated as “◯” in FIG. 1. The user handles by hand, the end of the test strip that is away from the reaction zones. An arrow marks the “horizontal” axis at which the user preferably holds the strip (i.e., he preferably holds the test strip in a horizontal position) to prevent possible cross contamination of chemicals (reagents) located in the adjacent reaction zones. This arrow and the term “horizontal” is not part of the test strip in FIG. 1. In another embodiment of the invention, the arrow and the term “horizontal” may be marked on the test strip so that the user can understand the position at which he should preferably hold the test strip.

FIG. 2 presents a non-limiting example of a color chart of the present invention. The color chart contains seven color codes and their explanations, respectively. Each color code is circular in shape and illustrated as “◯” in FIG. 2.

FIG. 3 presents a non-limiting example of a color chart of the present invention. The color chart contains seven color codes and their explanations, respectively. Each color code is circular in shape and illustrated as “◯” in FIG. 3.

FIG. 4 presents a non-limiting example of a color chart of the present invention. The color chart contains seven color codes and their explanations, respectively. Each color code is circular in shape and illustrated as “◯” in FIG. 4.

FIG. 5 presents a non-limiting example of a test strip of the present invention. The test strip is rectangular and contains seven reaction zones. Each reaction zone is circular in shape and illustrated as “◯” in FIG. 5. Under each reaction zone is marked (such as by printing) a number.

FIG. 6 presents a non-limiting example of a test strip of the present invention. The test strip is rectangular and contains seven reaction zones. Each reaction zone is in the shape of an Arabic numeral.

### DETAILED DESCRIPTION OF THE INVENTION

All publications and patent applications cited herein are hereby incorporated by reference in their entirety to the same extent as if each of them has been individually indicated to be incorporated by reference.

1. Shock and Dehydration

In physiology, “shock” is defined as insufficient oxygen supply to the body tissues and organs (heart, kidneys, intestines, brain, etc.). Blood delivers the oxygen, but there may not be enough oxygen in the blood, or there may not be enough blood (e.g., due to blood loss), or there may not be enough blood pressure to supply the organs with the amount of oxygen necessary to keep them alive and functioning. When an organism experiences shock, its organs (heart, kidneys, intestines, brain, etc.) are not getting sufficient oxygen. Early in shock, the organs function in a compromised state (the kidney produces less urine, the brain does not function as well and the organism becomes confused or lethargic), and later the organs cease to function (no urine output, coma set in), but the organs may still be revived with proper treatment of shock (this is similar to a plant without water; its leaves shrivel and wither, but with the return of water they may revive). In the final stage of shock, the organs suffer irreversible damage and no amount of oxygen-rich blood can return the organs to their normal state. In this case, the organism suffers multi-system organ failure and often death.

In dehydration, insufficient fluid in the body causes the cells to shrink and the fluid content of the blood to decrease. In this state, the organs are not working at their optimal capacity. The heart must work harder to pump the blood because there is less volume of liquid inside the blood vessel system. The heart works best when it has enough filling pressure and enough blood volume. The blood vessels to the kidneys constrict to try to conserve the remaining fluid in the system and the urine production decreases. The body prioritizes necessary functions and delays maintenance activities in the hopes that the body will find water and return to a normal state of hydration. In the normal state, the cells are full and plump and they are functioning in a relaxed state of good fluid supply. The enzymes and cellular functions are maximized. In the event of over-hydration, healthy kidneys can readily excrete the extra water in the urine. Severe dehydration is experienced as similar to blood loss. The small blood vessels constrict and the body tries to conserve the remaining fluid. The heart rate goes up and the blood pressure goes down. Both severe dehydration and blood loss are treated with infusion of fluid into the person's veins. This increases the volume of liquid in the blood vessels and allows the heart to pump more efficiently and the blood pressure increases.

In summary, shock is the condition whereby the body is not receiving enough oxygen delivery to the tissues. Shock can be due to blood loss, dehydration, or loss of blood pressure. Shock can also be caused by heart problems, insufficient blood volume, allergic reaction, infections, and damage to the nervous system. Among the possible treatments for shock are: fluid replacement with intravenous (“IV”) infusions of, e.g., balanced salt solutions; blood transfusions; and drug administrations (e.g., drugs to induce vasoconstriction). Shock is life threatening, because if left unchecked, it will cause organ failure and result in death. Unfortunately, shock can worsen and death can occur very rapidly without immediate medical treatment. Therefore, it is imperative that medical professionals be able to quickly diagnose that the patient is suffering from shock.

Unfortunately, the current art is lacking in a quick, easy and accurate (effective and efficient) means for diagnosing shock, particularly in its early stage. Clearly, the lack of a quick, easy and accurate test and the difficulty in determining whether a patient is in shock, are exacerbated in an emergency, such as in an emergency room, especially because the patient may have other critical conditions or injuries, and the speed at which shock can escalate into a life-threatening condition and death. The rapid assessment of shock is vital to the successful early treatment of trauma patients. In short, early identification and treatment of shock in trauma patients saves lives.

Currently, the diagnosis of shock depends on observing the following signs or symptoms of shock: (1) low blood pressure; (2) elevated heart rate; (3) low or no urine output due to inadequate blood flow to the kidneys; (4) pale, cool, clammy skin. Skin temperature in theory reflects the amount of blood flow. That is, warm and pink skin shows good blood flow and pressure, cold and clammy skin shows inadequate blood supply; however, the foregoing is often confounded by environmental exposure; and (5) reduced mental function, impaired speech, confusion or coma due to low blood flow into the brain. However, these diagnoses are not quick, easy and/or accurate. There is currently no efficient and effective way to measure the early states of shock.

For example, in the emergency room, the patient is typically assessed by: (1) the patient's vital signs. This test is an inaccurate reflection of shock in that it tends to overestimate the physiological stability of the patients, particularly of young patients. Many patients have few early signs of shock because of the natural compensatory mechanisms of the body. Once those mechanisms are exhausted, the patient is in dire circumstances and death can be rapid. Many patients may also present with normal blood pressure and heart rate, but later reveal serious injuries that are more difficult to treat with prolonged shock. This is also true of pre-operative and post-operative patients. See, e.g., C. V. Brown et al., Hemodynamically “stable” patients with peritonitis after penetrating abdominal trauma: identifying those who are bleeding, Arch. Surg. 140 (8): 767-72(2005), which concludes that patients with penetrating abdominal trauma or peritonitis require emergent operation regardless of their vital signs, because their normal systolic blood pressure and heart rate (i.e., hemodynamic “stability”) do not reliably exclude significant hemorrhage. Despite such “stability”, patients may suffer from vascular injury and subsequent hypotension, and require blood transfusion and complicated post-operative course.

The monitoring of the patient's vital signs is especially inadequate to indicate blood loss and shock in healthy young people, in particular children, because they tend to maintain normal vital signs until their bodies completely collapse and rapid death occurs. For these patients, there are few warning signs of shock because their bodies' compensation mechanisms work very well until their final collapse and rapid death. This effect is most profound in children who are able to maintain normal vital signs until they suddenly collapse into hypotension (low blood pressure that is inadequate to meet the needs of the tissues) and tachycardia (rapid heart rate, i.e., a sign of uncompensated shock);

(2) the patient's urine output. This test is also inaccurate because a patient may have adequate urine due to previously stored urine in his bladder. For example, the patient may have a substantial volume of urine in his bladder due to having previously drunk large quantities of beer before he got into a car accident and lost half of his blood. Thus, this kind of patient could not be assessed by the amount of urine in his bladder. Also, in order to be more accurate, the urine output would have to be drained from the bladder and new urine production measured over time after an injury for the low blood flow to the kidney to manifest itself. Generally, it would take at least about half an hour to manifest, and this would be too long for a patient in a critical condition. The measurement takes time because urine output is commonly quantified in thirty-minute time intervals. It is difficult to quantify in shorter time intervals since the urine volume is often too low to accurately measure; and

(3) the patient's skin temperature. However, this test is also inaccurate because skin temperature is strongly affected by the environment.

Other methods are invasive, time consuming, and/or unreliable. They include: (1) Gastric tonometry to measure the blood supply to the stomach. This requires a tube to be placed through the mouth or nose into the stomach, followed by the inflation of a balloon to measure the pressure of the stomach wall. It is uncomfortable for the patient and dangerous in a patient with a stomach full of food or beer. Also, it is often difficult for providers to insert the sizable tube; and (2) Taking of blood samples to determine hemoglobin and base deficit in order to assess blood loss. Hemoglobin measurement is wrong 50% of the time because acute losses of blood do not allow time for equilibration. Base deficit measurements require arterial blood sampling and are probably the best measure currently available for acute shock.

2. What are the Problems Solved by the Present Invention?

Clearly, a rapid, reliable, non-invasive method to diagnose shock and its extent, is a long-felt but unmet need. The present invention presents such a method by assessing the hydration status (end organ perfusion) of the patient. That is, applicant discovers that salivary amylase is an accessible end organ product, and is a reliable reflector of the hydration status (end organ perfusion) of the patient by quantifying the perfusion of one representative end organ (the salivary gland). Thus, the present invention presents the use of the patient's saliva sample to assay for the amount of salivary amylase to assess the status and extent of hydration and end organ perfusion of the patient, to diagnose shock and its extent, and/or to monitor the progress and/or resolution of hydration, end organ perfusion, and/or shock of a patient (for example, in order to assess the efficacy of his treatments and to determine whether some other factors are at play and so, some other treatments are necessary). For example, as the patient is being treated for shock, she can be monitored by periodic testing of her salivary amylase to determine her progress, and/or the efficacy of the treatment, and/or to alert the physician that other conditions (beyond which she is being treated) may be responsible for shock. This is useful in an emergency setting, etc.

Without wishing to be bound by her hypothesis, applicant hypothesizes that when a person is dehydrated or in shock, his sympathetic system is in a fight or flight state, and his blood selectively flows to the heart, brain and muscles but not to the digestive system. Applicant hypothesizes that since digestion is at a low priority when a person is dehydrated or in shock, his salivary amylase concentration will decrease. Salivary amylase concentration can be determined by, for example, assaying the person's saliva sample for salivary amylase activity using assays known in the art or modifications thereof, or those presented in the present invention. The salivary glands are organs that are affected by dehydration and shock just like other organs. When they do not receive enough blood flow (from dehydration, blood loss or shock), they stop producing their normal quantity of saliva and its digestive protein: salivary amylase. In the dehydrated or shock state, there is a lower concentration of salivary amylase in the mouth. When the dehydrated person drinks water or receives intravenous fluids, his salivary gland revives and returns to producing its usual (normal baseline) amount of amylase. Applicant also realizes that the amylase in saliva is a more real time indicator of the shock status of a person, then the amylase (whether salivary or pancreatic amylase) in his blood. This is because a person constantly swallows his saliva and so, the saliva in his mouth is freshly produced from which the saliva sample of the present invention is preferably obtained. By contrast, the amylase in blood could have been circulating for some time (prior to shock). Applicant's conclusion that the concentration of salivary amylase (from a fresh saliva sample) decreases with the onset and progress of shock is contrary to the teaching of the prior art {such as Nater, U M, et al., Stress-induced changes in human salivary alpha-amylase activity—associations with adrenergic activity, Psychoneuroendocrinology, 31(1): 49-58 (2006); and M. Yamaguchi, et al., Performance evaluation ofsalivary amylase activity monitor, Biosens. Bioelectron. 20(3): 491-7 (2004); discussed below} which found that a person under psychological stress has marked increase in salivary amylase concentration (from a fresh saliva sample). Applicant also concludes that the salivary amylase concentration in a person's saliva is related to his hydration status. That is, a person who is less hydrated will have less salivary amylase concentration in his saliva, and vice versa.

The present invention's method for assessing, quantifying, and/or monitoring the status and/or progress of dehydration and/or shock is rapid, reliable, painless, non-invasive, and has a low risk of infection. It is valuable for assessing the initial state of the subject and the evaluation of the effects of treatment (for example, drinking fluid or water, receiving an infusion of IV fluid or blood transfusion). If the responses to treatment are still inadequate then more aggressive treatments can be initiated (i.e., if the person is unable to drink enough to meet her needs, then an IV infusion must be started). The present method is so much faster, cheaper, easier, more convenient, and more accurate than that of the prior art that it can be used by physicians, nurses, and even the general consuming public.

This method is particularly helpful for young healthy people, especially children, since their bodies try to combat shock, compensate for it and disguise it (as discussed above), but this assay is a sensitive way to show that their bodies are using their compensatory mechanisms and need help to return to a normal healthy state. No other method currently available meets these criteria.

Since the salivary amylase assay is simple and convenient, it can be readily carried and used by the general public. For example, it may be used in non-clinical settings, e.g., wherein a user (or a third party) wishes to monitor her level and progress of hydration and end organ perfusion, for example, to maintain hydration in order to promote health or to avoid fluid loss which may escalate into shock or death. Also, in the field, the user can quickly use it to monitor and/or assess the dehydration and shock status of a person. Clearly, such simple, convenient assays are useful in a gymnasium or athletic facilities, in the field, etc., for athletes/sportsmen, outdoors men such as hikers and climbers, military personnel (in the battlefield or in exercise), outdoor workers (such as oilfield workers in the dessert or on oil rigs), and they are also useful for children engaged in physical activities, exposed to heat or the elements, etc.

The assays are also useful for detecting dehydration or shock status caused by diseases, such as infectious diseases, especially diseases that are not symptomatic at early stages. For example, young children and babies frequently become dehydrated especially when infected with a diarrhea-causing virus such as rotavirus, and they do not show early signs of the severity of dehydration. Young children and babies are often presented at the emergency room or a pediatrician's office late when they become lethargic (late stage of dehydration). Further, young children and babies are susceptible to dehydration from many different causes and it can be difficult for a mother, especially an inexperienced mother, to determine if her child is getting enough fluid. Wet diapers must be weighed to quantify how much urine output a baby has. This is a cumbersome and unreliable method. Thus, the medical professionals and consuming public (such as parents, caretakers, teachers, school, etc., of young children) may benefit from the present method. For a study of amylase activity of saliva from children attending a baby clinic, according to sex and age (for children aged 18, 30 and 42 months), see, C. C. Dezan, et al., Flow rate, amylase activity, and protein and sialic acid concentrations of saliva from children aged 18, 30 and 42 months attending a baby clinic, Arch. Oral Biol. 47(6): 423-7 (2002).

Also, the assays can be placed in containers (such as boxes) carrying supplies (such as bottles) of fluid as part of a medical or relief effort, for example, to a war-tom or disaster area or an underdeveloped country, where the victims are likely to be dehydrated or in shock due to diseases (such as cholera), environmental exposure or injuries.

3. Test Kits

As a non-limiting example, a test kit is provided which may include: a container of fluid (such as water, a drink containing balanced electrolytes, or a sports drink), and a salivary amylase assay that can be used to test the saliva of a test subject against a baseline to allow a user to determine whether the test subject is adequately hydrated. In yet another embodiment of the invention, the test kit provides different baselines to allow a user to determine whether the test subject is adequately hydrated, and if not, then the severity of dehydration. If the assay indicates that test subject is not adequately hydrated, he can then drink or be administered the fluid from the container of fluid. For example, this test kit can be sold by sports drink sellers to enable their consumers to assess their hydration level when exercising, e.g., in the gymnasium or outdoors.

In yet another embodiment of the invention, the test kit provides different baselines to allow a user to determine whether the test subject is adequately hydrated, and if not, the severity of dehydration, and the onset and progress of shock. If the assay indicates that test subject is not adequately hydrated, he can then drink or be administered the fluid from the container of fluid.

For emergency use, trauma use, or use in the battlefield or dangerous environment, the test kit can additionally contain anti-shock treatment materials, such as an emergency supply of blood for transfusion (which can include blood, blood products, blood components or artificial blood replacement products), intravenous fluids, and/or drugs (e.g., drugs to induce vasoconstriction), for administration to the test subject. The test kit can also contain equipments (needle, anesthesia, etc.) for blood or fluid infusion.

The test kit may also be provided only with the salivary amylase assay and/or means for reading or interpreting its results, without the container of fluid or emergency supplies. A non-limiting example of the means for interpreting the assay's results is a color chart as described in this application.

The test subject may or may not be the same as the user. For example, the test subject and user can be the same athlete or soldier. In another example, the user can be a supervisor or caretaker (such as a parent, teacher, coach, or military supervisor) or the user can be a medical personnel and the test subject is a person he serves (such as a child, a student, athlete, military personnel, patient). The user can be a caretaker (such as a pet owner) and the user is a pet, e.g., a pet engaging in physical activities.

The test kit may use any salivary amylase assay known in the art, or presented in the present application. Preferably, the salivary amylase assay is convenient to carry and use, and is accurate. For the average consumer, it is preferable that the salivary amylase assay indicates the levels of hydration by changes in colors (preferably different levels having corresponding different colors) that are visible to the naked eyes and easily understood by the user by means of comparing to a color chart (in which case, the color chart may be included with the test kit), such as the assays described in section “5. Test Strips”, below. If a test strip is used (see the discussion under section “5. Test Strips”, below), it is preferable that the test kit contains several test strips properly packaged (see the discussion under “Test Strips”, below) to allow the user to assess the progress of hydration and/or shock and accordingly take the appropriate actions.

For convenience sake, this application uses a patient as an example of a user of the invention. However, it is understood that the user can be any person. Further, this application uses a human as an example of a user. However, the present invention may be generally applied to animals that produce salivary amylase, of any sex. For example, the present invention may be used in veterinarian care or to monitor the dehydration of pets, such as when they are engaged in outdoor activities. It is noted that some mammals such as dogs, horses and cats do not produce salivary amylase.

4. Salivary Amylase and Its Assays

The present method relies on the determination of the amylase concentration in the patient's saliva by sampling his saliva. Methods known in the art, for collecting saliva sample from a person, may be used. For example: the patient may spit on a test-strip or aspirate onto a collecting tube; his saliva may also be collected through slight suction; a test strip may be placed under his tongue to collect the saliva, etc. Other collection methods are described further below. If a person is unconscious or incapacitated, is a senior or young child, his saliva may be collected by slight suction.

The assays are also being claimed. By using saliva sample, the present method for determining hydration and end organ perfusion is more accurate than an amylase assay using blood or serum sample. This is because the salivary gland constantly produces salivary amylase while the patient constantly swallows his saliva. Therefore, the salivary amylase concentration in the saliva is a more contemporaneous reflection of the hydration status of the patient than the amylase concentration in his blood or serum. This is because the salivary amylase in the blood or serum is likely to have been produced earlier and have been circulating in the blood or serum for some time.

Amylase is found in several organs and tissues of animals, including humans, plants and microorganisms. There are two forms of amylase: alpha and beta. Alpha-amylase cleaves the 1,4-alpha-glucosidically-linked polysaccharides to yield maltose (disaccharides of glucose) and malto-oligosaccharides. Beta-amylase is found in seeds before germination. In the present patent application, unless otherwise modified, the word “amylase” denotes “alpha amylase”. Amylase is a digestive enzyme. In humans, the pancreas synthesizes and secretes pancreatic amylase into the intestinal tract; and the salivary glands secrete salivary amylase that hydrolyzes starches (to produce maltose) while food is still in the mouth and esophagus. Maltose (a dimer of glucose) is then degraded by the enzyme maltase into glucose. Both pancreatic and salivary amylase are found in normal serum and urine. Diagnosis of various diseases (most commonly pancreatitis) can be made by quantitative analysis of pancreatic amylase in blood and urine. Amylase assay is easy to perform and has been the main test for pancreatitis. Laboratories generally measure either pancreatic amylase, or total amylase. Total amylase readings (in blood samples) of over ten-times the upper limit of normal (ULN) are suggestive of pancreatitis; between five- to ten-times the ULN may indicate ileus or duodenal disease or renal failure, and lower elevations are commonly found in salivary gland disease.

In one embodiment the present assay, the amount of amylase in the saliva sample can be determined indirectly by allowing the amylase to act on a system containing an excess and a predetermined amount of a substrate, and measuring the amount of maltose thus produced. For example, the sensor used in the U.S. Pat. No. 4,547,280 of Karasawa, et al., “Maltose sensor”, is an enzyme assay which can be modified and applied to the present invention to assay the salivary amylase in a saliva sample.

U.S. Pat. No. 4,337,309 of McGeeney, “Method of determining the concentration of pancreatic and salivary alpha-amylase in body fluids” explains that: It is known that many pathologic conditions cause changes in the amylase concentrations in body fluids such as serum and urine; and diagnostic methods for measuring the amylase concentration in body fluids have been developed. These methods are usually based on the degrading effect of amylase on starch products, the enzyme activity being determined by measuring, directly or indirectly, the degree of degradation of a starch-like substrate. Examples of such known methods are the“blue starch method” and the “saccharogenic method”. In the “blue starch method” the substrate is insoluble, cross-linked starch; whereas soluble (Zulkowsky) starch is used in the saccharogenic method. These and other methods for measuring the amylase concentration in body fluids are well known in the art. See e.g. O'Donnell and Mc. Geeney, Comparison of Saccharogenic and Phadebas.RTM methods for amylase assay in biological fluids, Enzyme 18, 348 (1974), and Robyt and Whelan in Starch and its derivatives, 4^{th} ed. J. A. Radley, ed, pp. 431-433 (Chapman and Hall, London 1968)—both cited by U.S. Pat. No. 4,337,309 of McGeeney, supra. Prior art pancreatic amylase assays, or amylase assays for use with serum or urine samples, that are based on amylase's enzymatic reaction may also be used in the present invention by modifying them for use with saliva samples. This is because both pancreatic and salivary amylases are alpha-amylases that react with their enzymatic substrate in the same way. On the other hand, salivary amylase may be differentiated from pancreatic amylase using monoclonal antibodies specific to one amylase but not to the other (see further discussion below). Thus, if a prior art immunoassay uses antibodies that do not differentiate between salivary amylase and pancreatic amylase, then the assay may be used for salivary amylase by applying it to saliva instead of non-saliva samples such as blood or urine. However, if the immunoassay uses antibodies specific to pancreatic amylase, then the assay (the present assay using saliva samples) should be modified to use antibodies specific to salivary amylase or antibodies recognizing both salivary and pancreatic amylase. This is because it is expected that either there is no pancreatic amylase in saliva or its presence is so small that it will not affect the validity of the assay result. Further, salivary amylase and pancreatic amylase behave differently on isoelectric focusing. Thus, the prior art assays that rely on the electric charge of the pancreatic amylase, should be modified to be specific for salivary amylase when used for the present invention.

The known enzymatic assay methods of amylase activity are based on the fact that a substrate glucose polymer such as starch is hydrolyzed by amylase action to form glucose, maltose or oligosaccharides. Examples are assay methods comprising measurement of the decrease of viscosity of starch by amylase action; iodometry; the reaction of glucose with glucose oxidase or glucose dehydrogenase and NAD (NADP), wherein glucose is formed by the action of alpha-glucosidase on maltose which is produced from amylase action on starch; and the blue starch method (described further below).

The known methods for quantitative analysis of amylase include the following general classes:
(1) An amyloclastic method for tracing gradual decomposition of starch by amylase according to iodine-starch reaction. An example of this method is the Caraway method that has been most widely used. Most colorimetric methods used for assaying the activity of amylase are based on its breakdown of starch. This reaction primarily yields intermediate dextrins and maltose. The rate of the reaction is usually monitored by (1) turbidimetric, (2) iodometric, and (3) reductometric methods. The iodometric method as modified by Caraway is considered one of the reference methods for amylase determination. An example of a commercial iodometric method (modified from the Caraway method) is the “Amylase (Colorimetric Method) Assay” commercially available from Teco Diagnostics, Anaheim, Calif. However, this Teco Diagnostic's assay uses serum sample. Thus, the present invention modifies the assay to apply to saliva samples as follows: saliva is incubated with buffered starch substrate at controlled temperature for 7.5 minutes and subsequently reacted with iodine to produce blue color with reacted starch. The decrease in color, compared with that obtained in the absence of amylase, provides a measure of amylase activity (which is one measure of amylase concentration). The amylase activity is determined by absorbance at 590 nm using a spectrophotometer. Amylase activities are calculated from the absorbance readings according to the instructions of the Teco Diagnostic's assay. The reagents are: (1) amylase substrate consisting of a solution of 0.04% starch, 0.85% sodium chloride, 0.86% benzoic acid, and 2.7% disodium phosphate, adjusted to pH=7.0; and (2) amylase color reagent consisting of a solution of 193 mM potassium iodide and 11.9 mM potassium iodate in diluted hydrochloric acid.
(2) A saccharogenic method for measuring the reducibility of maltose produced through decomposition by amylase. A typical example of this method is the Somogyi method, but there are problems in that since the measured value includes the value of glucose present in a sample and the blank value of sample should also be measured, the procedures are undesirably complicated, etc. Soluble (Zulkowsky) starch is used in the saccharogenic method;
(3) A chromogenic substrate method for colorimetry of soluble pigments freed from insoluble colored starch, as crosslinked with pigments, as a substrate under the action of amylase, etc. An example of this method is the generally used blue starch method or maltose phosphorylase method. The blue starch method is a widely used method that calorimetrically measures the soluble pigment (blue color) produced by amylase action on the insoluble pigment-bound starch (see, e.g., Japan. Pat. Publ. No. 55-27800, cited by U.S. Pat. No. 4,427,771 of Misaki et al., Assay method for amylase activity and method of producing maltose dehydrogenase for use therein and also cited by U.S. Pat. No. 4,683,198 of Ishikawa, et al., Novel maltose dehydrogenase, process for its production, and analytical method using the same. Both U.S. patents present amylase assays or reagents that may be used in the present invention). Filtration is essential for colorimetry, and so, an automatic procedure is not as feasible. In addition, other assays use a synthetic substrate for amylase. For example, a synthetic substrate p-nitrophenylmaltopentaoside can be reacted with amylase, liberating p-nitrophenyl by the action of alpha-glucosidase that is calorimetrically measured. In the prior art, this method is disadvantageous for assaying serum amylase because the reaction mixture is yellow, like bilirubin serum or hemolyzate. In contrast, this assay is useful for the present invention since saliva is not yellow. Another method uses the synthetic substrate (2,5-dichlorophenylmaltopentaoside) that is reacted with amylase, and further is treated with alpha-glycosidase and beta-glycosidase to liberate 2,4-dichlorophenol that is oxidatively condensed with 4-aminoantipyrine by sodium periodate, the rate of increase in absorbency is measured at 500 nm.
(4) enzymatic method: a maltose phosphorylase method comprising decomposing maltose, which has been produced from soluble starch as a substrate by amylase, by maltophosphorylase and ultimately measuring the amount of NADH (the reduced form of nicotinamide adenine dinucleotide) after three further enzyme reaction stages using beta-phosphoglucomutase, glucose-6-phosphate dehydrogenase, and 6-phosphogluconic acid dehydrogenase, respectively; and
(5) enzymatic method: an alpha-glucosidase method comprising decomposing maltose into glucose by alpha-glucosidase and assaying the glucose, etc. These enzymatic methods utilize the specificity of enzyme for substrates, and thus have the advantage of low susceptibility to influence by assay-interfering substances, as compared with methods (1) to (3), above, but have such disadvantages as a prolonged assay time, an impossibility to assay the whole blood, use of expensive analytical reagents such as enzymes and coenzymes, complicated structures of analytical instruments. To improve the assaying accuracy and simplify the operating procedure, an enzyme sensor method for assaying amylase has been recently proposed [K. Yoda and T. Tsuchida: Proceedings of the International Meeting on Chemical Sensors, p. 648 (1983), cited by U.S. Pat. No. 4,547,280 of Karasawa, et al., Maltose sensor]. This type of sensor is described in detail in U.S. Pat. No. 4,547,280 of Karasawa, et al., “Maltose sensor”.

Another enzymatic method is found in the “Amylase Reagent Set (Kinetic Method)” commercially available from Teco Diagnostics which uses the following chemical reaction: Amylase hydrolyzed p-Nitrophenyl D-Maltoheptaoside (PNPG7) to p-Nitrophenylemaltotriose (PNPG3) and Maltotetraose. Glucoamylase hydrolyzes PNPG3 to p-Nitrophenylglycoside (PNPG1) and glucose. Then PNPG1 is hydrolyzed by glucosidase to glucose and p-Nitrophenol, which produces a yellow color. The rate of increase in absorbance is measured at 405 nm and is proportional to the amylase activity in the sample. The Teco Diagnostics assay uses serum and urine samples. In the present invention, this assay is used on saliva sample. A salivary amylase assay using kinetic reaction and a chromogenic substrate on a 96-well microtiter plate is also commercially available from Salimetrics, LLC (State College, Pa., USA). All the foregoing assays and devices can be used, or modified (e.g., according to the teaching of this patent application or methods known in the art) to be used, in the present invention to quantify the amylase concentration (e.g., amylase activity) in a saliva sample;
(6) Recent salivary amylase assays: Recently, new salivary amylase assays and devices have been provided and they can also be used in the present invention. These assays include those using chemical reaction, antibodies, biosensors, etc. Examples of these assays and devices are described in, e.g.,
   (a) M. Yamaguchi, et al., Performance evaluation of salivary amylase activity monitor, Biosens. Bioelectron. 20(3): 491-7 (2004) [herein also referred to as “Yamaguchi et al (2004)”]; also by the same lead author, M. Yamaguchi, et al., Hand-held monitor of sympathetic nervous system using salivary amylase activity and its validation by driver fatigue assessment, Biosens. Biolectron. 21(7): 1007-14 (2006) [herein also referred to as “Yamaguchi et al (2006)”]. Both references are also herein collectively referred to as “Yamaguchi et al”. Yamaguchi et al (2004) finds that increase in stress levels could be shown by a rise in salivary amylase activity of the people tested. These references describe a hand-held monitor that is an automatic analytical system for salivary amylase activity using a dry-chemistry system, a disposable test-strip equipped with both collecting and reagent papers and automatic saliva transfer device. The collecting paper was directly inserted into an oral cavity and whole saliva collected from under the tongue, and the saliva immediately tested. The monitor uses the enzymatic reagent method with 2-chloro-4-nitrophenyl-4-O-β-D-galactopyranosylmaltoside (Ga1-G2-CNP), on a reagent paper, as a substrate for amylase. When Ga1-G2-CNP is hydrolyzed by amylase, the hydrolyzed product (CNP) develops a yellow color with time in the following reaction equation:
      Ga1-G2-CNP—amylase→Ga1-G2+CNP (white→yellow)
   The above enzymatic reaction continues until the substrate is completely consumed. The monitor is temperature- and pH-adjusted by using the equations that interpolate the values to 37° C. and pH 6.5. The references conclude that this hand-held monitor enabled a user to automatically measure salivary amylase activity with a high accuracy when only 30 μl of saliva sample could be collected, and within a minute from the time of collection to the completion of the measurement. Normalized equation was used to make individual variations of salivary amylase activity negligible. According to Yamaguchi et al (2006), p. 1009 (left col., last sentence), an international standard method for the measurement of amylase activity has yet to be defined. Yamaguchi et al (2004) concludes that its monitor can analyze salivary amylase activity without the need to determine the saliva volume qualitatively;
   (b) Nater, U M, et al., Stress-induced changes in human salivary alpha-amylase activity—associations with adrenergic activity, Psychoneuroendocrinology, 31(1): 49-58 (2006) (Herein also referred to as Nater et al)—A previous study by the authors found marked increases in salivary amylase following psychological stress. In this reference, salivary amylase was collected with a salivette. Amylase activity was determined using the automatic analyzer Cobas Mira and assay kits commercially available from Roche. The reagents in the kit contain the enzyme amylase and alpha glucosidase, which convert the substrate ethyliden nitrophenyl to p-nitrophenol. The rate of formation of p-nitrophenol is directly proportional to the amylase activity that is determined by absorbance at 405 nm. This is an example of a kinetic colorimetric assay;
   (c) Y. Noto, et al., The relationship between salivary biomarkers and state-trait anxiety inventory score under mental arithmetic stress: a pilot study, Anesth. Analg. 101(6): 1873-6 (2005);
   (d) G. H. Hoek, et al., Toothbrushing affects the protein composition of whole saliva, Eur. J. Oral Sci. 110(6): 480-1 (2002) (herein also referred to as Hoek et al)—this test uses monoclonal antibody. The assay is described on p. 480, right col., as follows: “Amylase activity was determined using the quantitative kinetic determination kit (no. 577 from Sigma Diagnostics, St. Louis, Mo., USA) as described by Henskens YMC, et al., Protein composition of whole and parotid saliva in healthy and periodontitis subjects; determination of cystatins, albumin, amylase and IgA. J. Periodontal Res. 31: 57-65 (1996)”;
   (e) A. O. Aluoch et al., Development of an oral biosensor for salivary amylase using a monodispersed silver for signal amplification, Anal. Biochem. 340 (1): 136-44 (2005)(herein also referred to as Aluoch et al)—this assay uses antibody. Its Abstract states thus: “An amperometric biosensor for monitoring the concentration of protein amylase in human saliva is described. A novel design and the preparation of amylase antibodies and antigens, essential for the development of biosensor, are reported. The biosensor sensing elements comprise a layer of salivary antibody (or antigen) self-assembled onto Au-electrode via covalent attachment. Molecular recognition between the immobilized antibody and the salivary amylase proteins was monitored via an electroactive indicator (e.g., K₃Fe(CN)₆) or a monodisposed silver layer present in solution or electrochemically deposited onto the solid electrode. The electroactive indicator was oxidized or reduced and the resulting current change provided the analytical information about the concentration of the salivary proteins.”;
   (f) A. Wolff, et al., Submandibular and sublingual salivary glandfunction in familial dysautonomia, Oral Surg. Oral Med. Oral Pathol. Oral Radiol. Endod, 94(3): 315-9 (2002)—the amylase activity was measured by means of a blocked paranitrophenyl glucose-7 method with a Raichem kit (RAI, San Diego, Calif.);
   (g) C. C. Dezan, et al., Flow rate, amylase activity, and protein and sialic acid concentrations of saliva from children aged 18, 30 and 42 months attending a baby clinic, Arch. Oral Biol. 47(6): 423-7 (2002) (Herein also referred to as Dezan et al)—amylase activity was determined by the method described by Fischer and Stein, Alpha-amylase from human saliva, Biochem. Preparat. 8: 27-33 (1961), as modified by Bellavia et al, Alpha-amylase activity of human neonate and adult saliva, Arch. Oral. Res. 24: 117-121 (1979), using a Beckman DB-G spectrophotometer. One enzyme unit is defined as the amount that catalyses the formation of 1 μmol of maltose under the specified condition.

In the present invention, the different degrees (levels) of dehydration (or hydration), onset of shock, and the different degrees of shock are directly related to the respective concentrations of amylase (e.g., as measured by amylase activity) in a saliva sample. The baselines (for the degrees of dehydration, onset of shock, degrees of severity of shock) for the salivary amylase concentrations can be derived, for example, by obtaining or monitoring the salivary amylase concentrations of patients upon admission to emergency room, as they are treated for dehydration or shock, and then correlating these concentrations with their subsequent determination of dehydration and shock and their severity, and their progress when treated for shock and dehydration. The known methods for correlating pancreatic amylase concentrations with acute pancreatitis or for other conditions may be used {see, e.g., D. J. Adam, et al., Serum amylase isoenzyme in patients undergoing operation for ruptured and non-ruptured abdominal aortic aneurysm, J. Vascular Surgery 30: 229-35 (1999); and studies related to commercial amylase assays for pancreatitis}, but in the present case, shock and dehydration are the conditions under study and saliva is the sample, and salivary amylase is tested. Statistical studies acceptable by the medical art are used.

The baselines (for the adequacy of hydration and degrees of dehydration) for the salivary amylase concentrations can be derived, for example, by using the method shown in Example 4, below, and based on a statistically acceptable pool of subjects.

There exist salivary amylase assays that are very sensitive and can detect salivary amylase activity down to a final concentration of 1×10⁻⁴ U/ml (0.001 units). Amylase concentration in human adult (men and women) saliva is reported to be 46±21 U/ml {see Table 1 of Hoek et al., Toothbrushing affects the protein concentration of whole saliva, Eur. J. Oral Sci 110: 480-481 (2002). Herein also referred to as Hoek et al}. Thus, as a starting point, in one embodiment of the present invention, an adult human is considered dehydrated if his salivary amylase activity is less than 46±21 U/ml as measured according to the Hoek et al assay or an assay producing results close to the Hoek et al assay. Hoek et al found that brushing did not significantly affect amylase concentration. One unit (U) is defined as the amount of enzyme required to liberate 1 mg of maltose from starch in 3 minutes at 20° C., at pH 6.9.

Notwithstanding the Hoek et al reference, the baselines (for the salivary amylase concentrations or salivary amylase activities corresponding to the normal or healthy state and to the different degrees of dehydration and shock) are preferably established according to the assay method for which the baselines are used, and according to parameters such as: age, sex, diet, and/or geographical locations (or any other parameters which may affect salivary amylase concentration or activity), to ensure that they are accurate. The methodology (including statistics) employed in the art may be used or may be modified to relate salivary amylase concentration or activity with the foregoing parameters. For example, Dezan et al relate salivary amylase activity to the age and gender of the test subjects by studying babies and children. See, C. C. Dezan, et al., Flow rate, amylase activity, and protein and sialic acid concentrations of saliva from children aged 18, 30 and 42 months attending a baby clinic, Arch. Oral Biol. 47(6): 423-7 (2002)(herein also referred to as Dezan et al). For example, Dezan et al's Table 2 shows the salivary amylase activity of children according to their ages (18, 30 and 42 months, respectively) and sex—the children have about 9, 12 and 10 μmol maltose/mg protein at ages 18, 30 and 42 months, respectively. Thus, as a starting point, in one embodiment of the present invention, a child is considered dehydrated if his salivary amylase activity is less than 9, 12 and 10 μmol maltose/mg protein at ages 18, 30 and 42 months, respectively, as measured according to the Dezan et al assay or an assay producing results close to the Dezan et al assay. See also, Hoek et al, and Yamaguchi et al, discussed in the present patent application, which present the salivary amylase activity of their test subjects before their tests (tooth brushing in the case of Hoek et al, and stress or fatigue tests in the case of Yamaguchi et al.). Thus, this pre-test salivary amylase activity is the “normal” baseline for their test subjects. In the case of the present invention, the “normal” baseline would be the salivary amylase concentration (e.g., as expressed by amylase activity) of a fully hydrated and healthy test subjects (not under shock, nor dehydrated)—this baseline can be the average of a statistically representative population of test subjects.

Based on the above, one skilled in the art would be able to devise other assay systems using the principles described above, to quantify, i.e. determine the concentration of, the amylase in the saliva sample of a test subject, and to correlate the concentration to the hydration status and degrees of dehydration and shock of the test subject. The test subject is preferably a human.

5. Test Strips

Two non-limiting examples of a test strip are: (1) the test strip has an antibody to salivary amylase with a color indicator that changes color with contact with salivary amylase. This strip would have several different sections or “wells” (“reaction zones”) that allow for different color indicators of varying amylase concentrations; and (2) a carbohydrate impregnated strip that provides substrate for the amylase that naturally occurs in saliva. After a certain period of time has elapsed, the remaining carbohydrate is developed with an iodine-based reagent. The latter is a separate liquid that must be dropped onto the strip with an eyedropper or other form of pipette or syringe.

The assay of the present invention is provided in various physical embodiments, including test kits and test strips. A test kit usually include all the reagents used in the assay methods described herein and the saliva sample is added to the reagents, which may be in a liquid system or a physical system like filter paper, etc. The substrate may be added to the system together with the saliva.

This section describes one embodiment of the invention: the test strip. Typically, a test strip will be prepared by impregnating an absorbent material with solutions containing the reagents necessary for the corresponding determination. Suitable absorbent carriers for the test strip include all the inert absorbent carriers used in the art. Most widespread is filter paper, but other absorbent cellulose or synthetic resin products may be used.

Thus, one example of the assay comprises a test strip, a dipstick, or swab (hereinafter collectively termed “test strip”) that can be contacted with fresh saliva. The test strip can be rubbed in the mouth, a person can spit onto it, or he can hold it in his mouth to wet it, etc.

In a non-limiting example, the test strip may provide for a rapid, colorimetric assay for the detection and/or monitoring of the hydration status or shock status of an animal that produces salivary amylase, by testing the saliva sample of the animal, wherein:
(1) the test strip contains a series of spaced-apart reaction zones; and
(2) the reaction zones contain: (a) reagents providing multiple biochemical pathways for the salivary amylase in a saliva sample to convert a substrate (such as carbohydrate or its synthetic substitute) on the reaction zones, to glucose or maltose, and (b) reagents for the colorimetric detection of the resulting glucose or maltose so as to provide visually detectable readout in order to provide visual determination of the concentration of salivary amylase in the saliva sample, the hydration level and/or shock level of the animal.

In the above example, amyloclastic, saccharogenic, chromogenic, enzymatic, colorimetric reagents, and/or other reagents known in the art, and modifications thereof, may be used (such as those discussed in the present patent application).

In another non-limiting example, the test strip may provide for a rapid, calorimetric assay for the detection and/or monitoring of the hydration or shock status of an animal that produces salivary amylase, by testing the saliva sample of the animal, wherein:
(1) the test strip comprises a series of spaced-apart reaction zones; and
(2) the reaction zones contain: (a) reagents providing multiple monoclonal antibodies capable of binding salivary amylase in a saliva sample, and (b) reagents for the calorimetric detection of the resulting “monoclonal antibody-bound-salivary amylase” to provide visually detectable readout in order to provide visual determination of the concentration of salivary amylase in the saliva sample, the hydration level and/or shock level of the animal.

In the above example, antibodies and/or colorimetric reagents, and other immunoassay or immunochromatographic assay reagents known in the art or modifications thereof may be used (such as those described in the present patent application). The following describes further examples of a test strip.

The test strip Rapignost-Amylase (commercially available from Behring) is used for the rapid determination of amylase in the urine. In their abstract, Troger et al find that the Rapignost-Amylase test strip is also suitable for the determination of salivary amylase in stains stored up to 6 weeks at room temperature. The stains are extracted with physiological saline and the application zone (“reaction zone”) of the strip is wetted with the extract. Positive amylase-reaction is recognized as a reddish-violet coloration of the reaction zone. Biological stains with low amylase concentrations react amylase negative. The method is uncomplicated and can be completed within 30 minutes. The test strips are easily available and stable during storage. Troger et al finds that the procedure is suitable for determining salivary stains for use in forensic biology. {H. D. Troger et al., Detection of saliva traces using test strips, Forensic Sci Int. 25(2): 143-6 (1984). Herein also referred to as Troger et al}. Kemppainen, et al use urinary dipstick test for amylase to diagnose acute pancreatitis in emergency setting. Kemppainen, E. A., et al., Rapid Measurement of Urinary Trypsinogen-2 as a Screening Test for Acute Pancreatitis, New Eng. J. Medicine, 336: 1788-1793 (1997). Hedstrom et al develops a rapid urinary test strip for detecting pancreatic amylase to screen for acute pancreatitis. The test strip is based on the immunochromatography principle and uses two monoclonal antibodies specific for pancreatic amylase. J. Hedstrom, et al., Evaluation of a new urinary amylase test strip in the diagnosis of acute pancreatitis, Scandinavian J. Clin. Lab. Investigation, 58(8): 611-616 (1998), herein also referred to as Hedstrom et al. Thus, one embodiment of the present invention use the foregoing test strips on saliva sample (instead of urine) to detect and quantify salivary amylase (instead of pancreatic amylase) as an indicator of hydration and shock status of a person (instead of pancreatitis). The reagents used in the present test strip would be for salivary amylase, or amylase in general because it is expected that the amount of non-salivary amylase in saliva will be minimal such that its effect on the assay result will be di minimis. For example, the above Hedstrom et al test strip would be modified to use monoclonal antibodies specific for salivary amylase (instead of pancreatic amylase) or specific for both salivary and pancreatic amylases. Such monoclonal antibodies are known in the art and commercially available. For example, one can use the antibody against salivary amylase of the SYNCHRON system of Example 1, below. The monoclonal antibodies against salivary amylase can also be obtained using methods known in the art, such as described in U.S. Pat. No. 4,863,728 of Gerber et al., Monoclonal anti-alpha-amylase antibody which non-specifically inhibits the enzyme activity of human alpha-amylase, which presents monoclonal antibody to human salivary amylase which inhibits enzyme activity of both human salivary amylase and human pancreatic amylase; see also, the other publications cited in the present patent application, such as A. O. Aluoch et al., Development of an oral biosensor for salivary amylase using a monodispersed silver for signal amplification, Anal. Biochem. 340 (1): 136-44 (2005)(herein also referred to as Aluoch et al), discussed above. Where necessary, the sensitivity of the test strips may be increased by increasing its reagent concentration, if saliva collection is found to be too small in volume compared to urine collection. An example of the physical structure of a test strip is found in United States Patent Application 2004/0028608A1 of Saul et al., Hygiene monitoring, e.g., its FIGS. 4, 5 and 7. The foregoing test strips may be modified or changed using skill known in the art, for example, by using different reagents, reaction formats, and materials for the test strips.

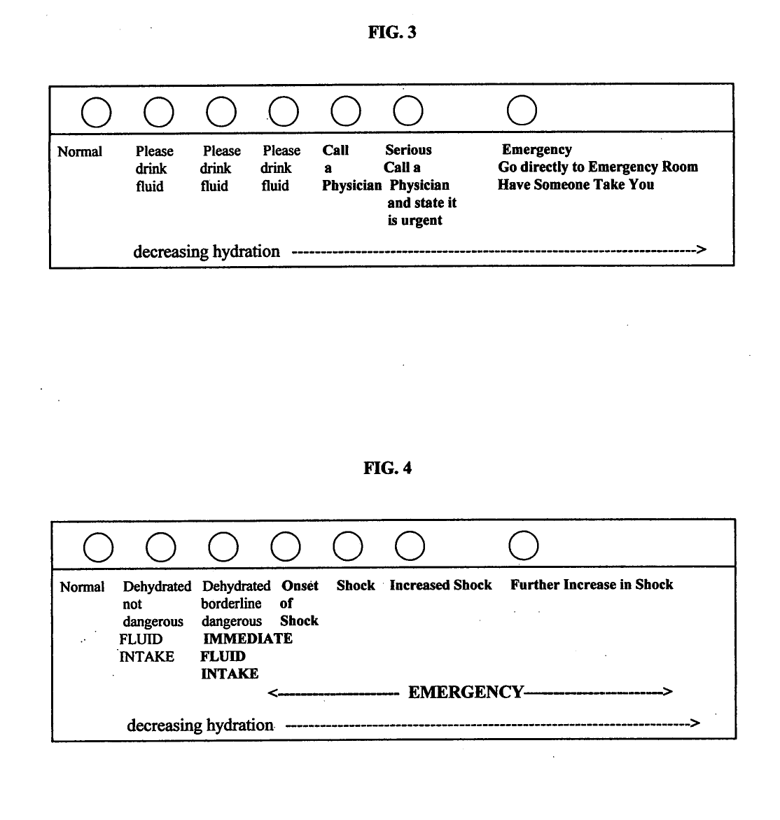

In one embodiment of the present invention, the test strip has the characteristics found in commercially available urinalysis test strips, such as URS-K (to assay for Ketones in the urine), URS-3 (to assay for Glucose, Protein, pH) and URS-10 (to assay for Glucose, Protein pH, Leukocytes, Nitrites, Ketones, Bilirubin, Blood, Urobilinogen, and Specific Gravity) that are used by the medical establishment or commercially available over the counter (commercially available from, e.g., Teco Diagnostics, Anaheim, Calif.). An example of the present test strip is a plastic strip to which reagent pads (serving as the “reaction zone”) specific to amylase or salivary amylase are affixed. The test strip may also be a paper to which several separate reagent pads are affixed. The reagents react with the saliva sample to provide a standardized visible color reaction in preferably a short time (preferably within seconds or minutes). One or more of the test strips may be sold in a test kit (see the discussion under the section “Test Kit”, above). The color is then visually compared to the color chart (preferably supplied with the strips) to determine the concentration of the salivary amylase. The test results provide useful information about salivary amylase, hydration status, and/or shock status of the person tested. The results may be expressed on the color chart as: (1) adequately hydrated or not; (2) in shock or not; (3) varying degrees of hydration; (4) varying degrees of shock; and/or (5) varying amylase concentrations or activities. Some of the foregoing are illustrated in FIGS. 2 to 4.

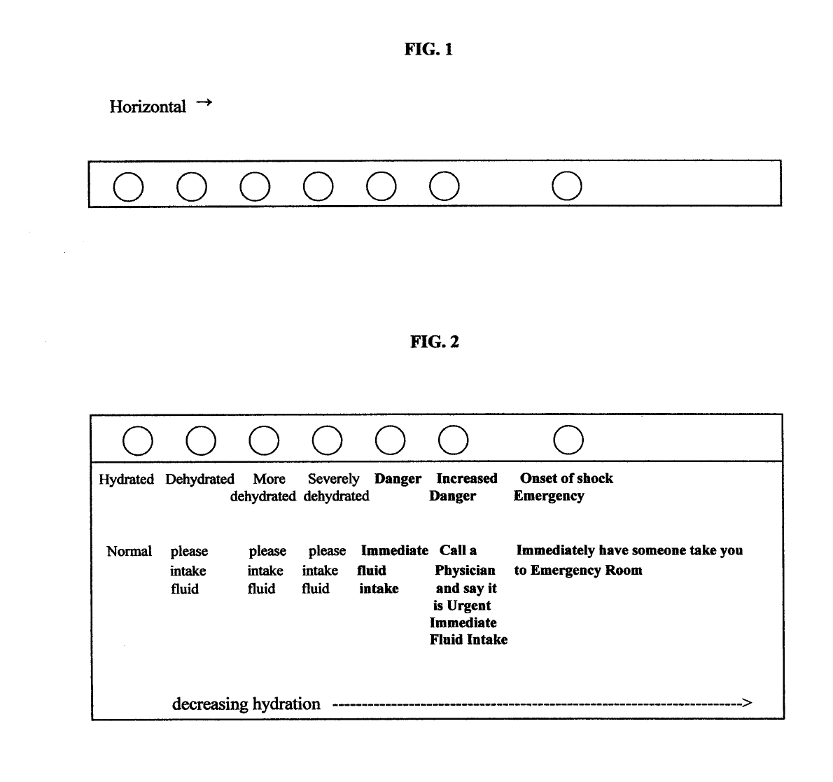

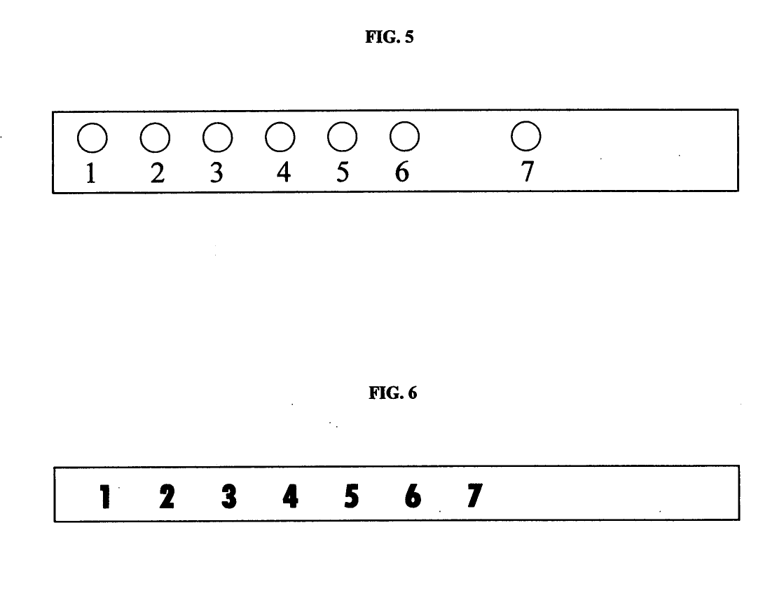

Non-limiting examples of a test strip are shown in FIGS. 1, 5 and 6. In FIGS. 1 and 5, even though each of the reaction zones is shown as circular, the reaction zone can be of any shape. Preferably, for the convenience of the visual differentiation by the user, each reaction zone produces a different color (from the other reaction zones) upon reacting with its concentration of salivary amylase. In an alternative embodiment, for the convenience of a color-blind person or for better visualization under certain circumstances, the test strip may be designed such that each reaction zone produces a change in color intensity, such as from a light shade of red to dark red (as opposed to a color change, such as from white to purple) when it reacts with a specific concentration of amylase (for amylase quantification and assessment of the corresponding hydration status of the test subject). Thus, in the discussion herein (e.g., the discussion below), it is to be understood that the changes in color can be expressed in at least two different alternatives: changes from one color to a different color, or changes to a different intensity (shade) of the same color.

The user can then match the color to the corresponding color chart for his interpretation. FIGS. 2 to 4 present non-limiting examples of the color chart. In FIGS. 1 to 4, seven reaction zones on the test strip of FIG. 1 and their corresponding seven color codes on the color charts of FIGS. 2 to 4 are provided. One skilled in the art would understand that he can use fewer or more of the reaction zones or color codes, in order to tailor them to his use. For example, if he wants to determine more degrees (gradations) of salivary amylase concentrations, hydration or shock status, he can have more reaction zones and corresponding color codes. The color codes may also reflect salivary amylase concentrations, hydration status, and/or shock status. In another embodiment, the different reaction zones generate the same (instead of different) color change, and the user match the test strip to the color chart by the locations of the reaction zones. Even though FIGS. 1 to 4 show the reaction zones and the corresponding color codes in a single row, it is understood that the reaction zones and its corresponding color codes may be arranged in different formats, such as circular, in several rows, etc.

The following presents non-limiting examples of the test strip and color code:
I) At the start, before the test strip is exposed to the saliva sample:
(1) In one embodiment of the invention, all the reaction zones on the test strip have the same color as that of the test strip. For example, the reaction zone can be transparent patches affixed to the test strip or the reagents are added to the test strip to create a reaction zone. The reagents can be so microscopic or be in a solution that is dried out on the reaction zone such that they do not affect the color of the test strip. Alternatively, the reagent patches are transparent, or the reagents and patches are of the same or similar color as the test strip, etc. The reaction zone's boundary is marked by print or ink or some manner visually perceptible; or
(2) In another embodiment of the invention, all the reaction zones on the test strip have the same color, but not the same color as the test strip. That way, the user can see where the reaction zones are and to make sure she applies saliva to those reaction zones; or
(3) In yet another embodiment of the invention, the test strip uses any other known means or modifications thereof for providing the reaction zones and their color coding.
II) Then the test strip and its reaction zones are contacted with the saliva sample, after the saliva has sufficient time to react with the reagents in the reaction zones:
(1) For the test strip using reaction zones which have the same color or different color than the test strip: Only the reaction zone which shows the correct concentration of salivary amylase, acquires a color visually different from that of the test strip and the original color of the reaction zone. All the other reaction zones maintain their original color. So the user uses the color as a guide to match the location of this reaction zone to the color code (e.g., on the color chart of either FIGS. 2, 3 or 4) that most closely corresponds to the correct concentration of salivary amylase. This can also be used by a color blind person;
(2) For the test strip using the reaction zones which have the same color or different color from that of the test strip: The reaction zones may change to the same color, but at different intensities (shades) of the color. The reaction zone with the most intense color is the one that most closely corresponds to the correct concentration of salivary amylase. This can also be used by a color blind person. For example, three reaction zones can change to pink, red, dark red, respectively (corresponding to increasing concentration of salivary amylase). After reacting with a saliva sample, if only the first two reaction zones change to pink and red, respectively, and the last reaction zone retain its original color (which is visually different from pink, red or dark red), then the user matches the red color to the corresponding salivary amylase concentration;
(3) In another embodiment, the test strip has only one reaction zone, then the new color acquired by the reaction zone or the intensity of the new color, is matched against the color chart for the corresponding salivary amylase concentration. For example, the color changes from white (original color of reaction zone) to red, and the intensity of the red color is matched to the color code to determine the most closely corresponding concentration of salivary amylase. Alternatively, the white color may change to red, blue, or yellow. Each new color corresponds to a particular concentration of salivary amylase;
(4) Yet another embodiment of the invention uses any other modes of visual differentiation used by test strips known in the art (e.g., commercially available test strips for testing ketone, etc.), or modified using prior art knowledge.

Yet another embodiment of the invention is a modification of FIG. 1, and is shown in FIG. 5. In FIG. 5, under each circular reaction zone, there is a specific number assigned to the particular reaction zone. As shown in FIG. 5, the seven reaction zones are numbered from 1, 2, etc., up to 7, respectively. Alternatively, each reaction zone may be shaped according to a specific number: 1, 2, 3 etc., up to 7, as shown in FIG. 6. In FIG. 6, the reaction zones have the same color among themselves but a different color from that of the test strip. It is understood that the reaction zones can have the same color as that of the test strip, as discussed above. After sufficient time has elapsed for the saliva sample to have reacted with the reaction zones, then for example, for moderate dehydration numbers 1, 2 and 3 reaction zones change color, for normal hydration all seven numbers for the seven respective reaction zones change color, and for severe dehydration only number 1 reaction zone changes color. In this embodiment, no color chart is required. Instead, a chart or explanation may be provided that relates the hydration status to the numbers. It is understood that the foregoing embodiment may be varied or modified to produce other embodiments, such as by using other patterns of color changes (e.g., as described in previous paragraphs), that more or less number of reaction zones may be used, different numbers may be assigned to different hydration status, or the Arabic numerals may be replaced with other numerals such as roman numerals, the alphabets or any other designations known in the art. It is believed that the designs of the test strips (of FIGS. 5 and 6 and their alternatives) are novel and it is envisioned that these designs can be used to assay for other analytes, besides salivary amylase, by using the appropriate reagents and assay conditions.

Preferably, the dimensions of the test strip are such that the test strip is convenient to handle by hand, convenient to insert and retrieve from the mouth for collecting a sufficient amount (for amylase detection or quantification) of saliva by hand, and allow easy visual observation of the color changes in the reaction zone(s) of the test strip. Non-limiting examples of the dimensions for the test strip are: about 0.25 inches in width and about 3 inches in length; the dimensions used by the salivary amylase test strip Rapignost-Amylase (commercially available from Behring and was studied by Troger et al, Detection of saliva traces using test strips, Forensic Sci Int. 25(2): 143-6 (1984), discussed above); and the dimensions used by the test strip of Yamaguchi et al 2004 and Yamaguchi et al 2006.

Yamaguchi et al 2006 used the following: a hand-held monitor consisted of a disposable test-strip and a monitor (110 mm×100 mm×40 mm); the test strip consisted of a sleeve, a sheet, a collecting paper (10 mm×10 mm×0.25 mm, 23 microliter) and a reagent paper for amylase (4 mm×4 mm×0.25, 4 microliter); the collecting paper was directly inserted into an oral cavity and approximately 20 to 30 microliter of whole saliva was collected from under the tongue. Unlike Yamaguchi et al, the test strip of the preferred embodiment of the present invention already contains the reagents for amylase in its reaction zones, therefore, an additional reagent paper is not required. Preferably, the present test strip has also been buffered to make the effects of salivary pH negligible.

The test strips may be stored in a container (such as a vial) and are ready to use upon removal from the vial and the entire test strip is disposable. The test strips may also be individually wrapped (e.g., in foil, paper or plastic). Each test kit may come with a complete abstract that includes a color chart for rapid visual diagnosis that explains the test and the color chart and what each color indicates. The reactive color of each reaction zone on the test strip is compared to the closest corresponding color on the color chart. In one embodiment of the invention, the concentration range for the salivary amylase is indicated below each color code on the color chart. As with all tests dealing with color intensity or color matching, it is recommended to obtain another person's interpretation of the test results. Preferably, the color chart [or the abstract found in the test kit] also shows the reader the color that identifies the normal (well hydrated) status, and the colors that identify the different degrees of abnormal (dehydrated) states. Preferably, for those test strips for use with detecting shock, the color chart or the abstract also show the reader the color that identifies the normal (no shock) state, the color that identifies shock onset, and the colors that identify the different degrees of shock.

One example of the test strip with reaction zones is shown in FIG. 1, the procedure for using the test strip is: (1) remove the strip from its container or wrapping, (2) collect a fresh saliva sample by spitting onto or placing in the mouth the test strip, to completely moisten the reagents on the test strip; (3) hold the strip in a horizontal position to prevent possible cross contamination of chemicals (reagents) located in adjacent reaction zones (see FIG. 1); (4) after holding the strip for the time listed on the color chart (or abstract) as necessary to complete the reaction, compare the color change of the reagent zone to the corresponding color chart. Non-limiting examples of the color charts are shown in FIGS. 2 to 4. Read the results according to the chart; (5) if need be, record the results of the readings for discussion or evaluation with medical personnel and then discard the test strip.

It is envisioned that the test strips may use any of the assay methods described in the present patent application, or their modification that are within the skilled person in the art, whether they are based on immunochromatography, immunoassay, amyloclastic, saccharogenic, chromogenic substrates, enzymatic or biosensor methods, etc., so long as they show color changes that can be visibly detected by a person.

6. Colored Test Strips

For the convenience of illustrating the test strips, the following discussion uses a saliva sample as an example of a sample. However, samples other than saliva, for which one wishes to detect or measure the level of amylase, may also be used.

Another embodiment of the invention presents a test strip that includes colored reaction zones (e.g., “reaction wells”) that consist of carbohydrate or sugars (or generally, amylose substrate) attached to an indicator molecule.

In one embodiment of the test strip, the test strip contains a gel matrix material consisting of agar and a blue-dyed amylose substrate. When alpha-amylase is added to the matrix, the dye will be cleaved from the substrate, leaving behind a white area on the matrix.

The test strip maker can control how much of a clear zone appears by adding defined amounts of enzyme to isolated sections of the matrix.

Several amylose-dye conjugates could be injected into the matrix at varying concentrations so that different levels of amylase leave behind different colors on the test strip. For example, a test strip may have 10% red substrate and 90% blue substrate. This strip will have a mostly blue appearance at the start—before a saliva sample is applied to the test strip. A saliva with low amylase activity (e.g., low concentration of amylase) will consume primarily the blue substrate which is present in abundance. The test strip might now take on a more purple appearance as the relative concentration of the red substrate increases. A saliva sample with high amylase activity (e.g., high concentration of amylase) will consume all the substrate, and leave behind a white area. For example, three types of colored conjugates (blue, red, orange) that are available (e.g., Azurine-crosslinked polysaccharides for the assay of endo-hydrolases which are available from the company Megazyme; and Ten et al., Development of a plate technique for screening of polysaccharide-degrading microorganisms by using a mixture of insoluble chromogenic substrates, J. Microbiological Methods 56: 375-382 (2004)] may be used for this test strip. As far as accuracy and the number of value ranges—besides a discrete step-wise changes in color, the test strip may alternatively have gradual shifts from one color to the next, which will allow for relative quantitation when compared with a color key. The color key may be provided with the test strip, such as provided on the side of the container for the test strips.

In an alternative embodiment, the test strip may have different matrix zones, each with a different substrate, resembling a pH paper in that the test strip will change to a different color to reflect different concentration levels of amylase in the sample tested.

Furthermore, the previously discussed embodiments of the test strips may also have one or more colored reaction zones (rather than colorless or opaque reaction zones that react with amylase and acquire colors) that are present before the testing, and the color disappears or changes color when a test sample containing amylase is added, to reflect the presence, concentration or level of amylase in the sample.

In one embodiment of the test strip, the test strip contains a colored positive control reaction zone, a colored negative control reaction zone, and three colored reaction zones for detecting three different concentrations of amylase. The user applies his saliva sample to all the reaction zones. He will know that the reaction is complete when the color disappears or changes the designated color in the positive control reaction zone.

Note that certain patients, such as people with intrinsic salivary dysfunction such as Sjogren's syndrome or other diseases of the salivary glands are not appropriate subjects for the test strips.

Having described the invention, the following examples are presented to illustrate and support the invention, and are not to be construed as limiting the scope of the invention.

### EXAMPLES

### Example 1

### Chemical Assay For Salivary Amylase

There are commercial assays for blood samples. For the clinical analysis of hyperamylasemia, it is important to assay for only the pancreatic amylase in the blood sample, and to exclude the salivary amylase that is also present. Various methods have been proposed for excluding the salivary amylase, these methods include: (1) separation by use of difference in charge (electrophoresis is used in clinical examination), (2) a gel filtration method, (3) an affinity chromatographic method, (4) an immunological method, (5) applying salivary amylase inhibitors (this method is used in clinical examinations).

In yet another example, amylase activity in blood and urine shows a remarkable increase in the case of pancreatic injury, pancreatitis, cancer of the pancreas, and parotitis when compared with normal values. In the commercial assays, serum samples are used and the assays must isolate the pancreatic amylase from the salivary amylase also present in the serum samples. The foregoing is accomplished by inhibiting salivary amylase activity, e.g., by using monoclonal antibodies specific to salivary amylase but not pancreatic amylase (see, e.g., the SYNCRHON System described below).

Since the assay of the present invention uses saliva sample and saliva contains only salivary amylase (or the amount of non-salivary amylase is so small that it is not expected to substantially affect the accuracy of the assay result), the present assay is simpler and does not suffer from any artifact that may be caused by the interference of other amylases, such as pancreatic amylase. Also, saliva collection is non-invasive, easy and convenient, as compared to the collection of other samples, such as serum. Examples of the present assay can be based on the modification of existing assays of serum samples, but without the extra steps and reagents necessary to inhibit the non-salivary amylase. Thus, the present method is easier, simpler, cheaper and more convenient than that of the prior art assays. The following is a non-limiting example of how a prior art assay may be modified to become the present assay.

Commercial Assay for Pancreatic Amylase: The SYNCHRON® Pancreatic Amylase (PAM) system (“SYNCHRON system”) is commercially available from Beckman Coulter, Inc., Brea, Calif. The SYNCHRON system uses serum samples to assay for specifically pancreatic amylase and to specifically inactivate/exclude salivary amylase. The pancreatic amylase assay is used to diagnose acute pancreatitis.

In the SYNCHRON system, pancreatic amylase reagent measures pancreas-specific amylase by an immuno-inhibition method. In the first incubation step, human salivary amylase activity is inhibited by two monoclonal antibodies that do not affect pancreatic amylase. After a second incubation with the substrate, the pancreatic amylase cleaves the substrate (4,6-Ethylidene-G₇-p-Nitrophenol) into fragments and these fragments are further hydrolysed by alpha-glucosidase to yield p-nitrophenol and glucose (See Table 1, below). The SYNCHRON system automatically proportions the appropriate serum sample and reagent volumes into a cuvette and monitors the change in absorbance at 410 nm. This change in absorbance is directly proportional to the activity of amylase in the sample and is used by the SYNCHRON system to calculate and express the pancreatic amylase activity. For further details, see the manual for the SYNCHRON system and its Bulletin 9282 s4, copyright 2003, Beckman Coulter, Inc., herein incorporated by reference in their entirety.

The SYNCHRON system's chemical reaction scheme (disclosed in its Bulletin 9282 s4, copyright 2003, Beckman Coulter, Inc.) is as follows:

TABLE 1
SYNCHRON System-Chemical Reaction Scheme:

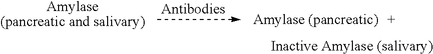

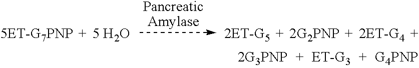

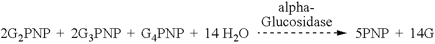

An Example of the Salivary Amylase Assay of the Present Invention: In this example, the present assay uses the commercial SYNCRHON System, but with the following modifications: Unlike the SYNCHRON System, the present assay uses saliva instead of a serum sample. Since there is no non-salivary amylase in saliva (or no significant amount of non-salivary amylase is expected so as to substantially affect the assay result), the present assay does not use the monoclonal antibodies to inhibit the non-salivary amylase, thus skipping the step of the SYNCRHON System that uses the monoclonal antibodies (in the case of the SYNCHRON System, this is to inhibit the salivary amylase), and the amylase being assayed for is the salivary amylase. The rest of the SYNCRHON System applies. The simpler chemical reaction scheme of the present invention is shown in Table 2, below.

TABLE 2
The Chemical Reaction Scheme of
One Example of the Present Invention

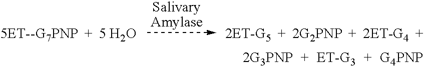

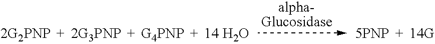

Other Chemical Assay Systems for the Present Invention: From the above example, one skilled in the art would be able to devise other chemical assay systems using the chemical reaction scheme of Table 2 and its reagents.

### Example 2

### Assay for Determining Hydration Level of a Person

25 μL saliva sample (test sample) collected from a human is added to 1000 μL of a commercially available reagent for the determination of amylase with 4-nitrophenylmaltohaptaoside (Boehringer Mannheim, Cat. Order No. 568589) according to the procedure in the manufacturer's instructions. The amylase activity of the test sample is determined at 37° C. according to the manufacturer's instructions. The controls contain varying amylase activities (corresponding to different hydration levels) are also determined according to the manufacturer's instructions. That is, the controls may be derived from the test protocols of Example 4, below, but using 4-nitrophenylmaltohaptaoside (Boehringer Mannheim, Cat. Order No. 568589) in the amylase assay according to the procedure in the manufacturer's instructions and the amylase activity is determined at 37° C. according to the manufacturer's instructions. The test sample's salivary amylase activity is compared to those of the controls. The control most closely matching the test sample is noted, and the hydration level of this control most closely matches the hydration level of the test human.

### Example 3

### Assay for Determining Hydration Level of a Person

50 μL saliva (test sample) collected from a human is used in a commercially available amylase test with blue-colored, high polymer starch substrate (Pharmacia Diagnostics AB, Uppsala, Sweden, Catalogue Order No. 93-986-2-1393-02) according to the manufacturer's instructions, and determined at 37° C. The controls contain varying amylase activities (corresponding to different hydration levels) are also determined according to the manufacturer's instructions. That is, the controls may be derived from the test protocols of Example 4, below, but using the commercially available amylase test with blue-colored, high polymer starch substrate (Pharmacia Diagnostics AB, Uppsala, Sweden, Catalogue Order No. 93-986-2-1393-02) according to the manufacturer's instructions, and determined at 37° C. The test sample's salivary amylase activity is compared to those of the controls. The control most closely matching the test sample is noted, and the hydration level of this control most closely matches the hydration level of the test human.

### Example 4

### Correlating Hydration with Salivary Amylase Concentration to Confirm that Salivary Amylase Concentration is Correlated with Hydration Status and Shock of a Test Animal

The subjects are ten healthy adult men and ten healthy adult women over the age of 18 years, without oral or systemic diseases, and not under medication. They are supplied with plenty of fluid. To ensure that the subjects are fully hydrated, their urine output is verified at greater than 1 cc/kg/hour; their saliva samples are then collected and their salivary amylase concentrations established using a salivary amylase assay known in the art (preferably a commercial assay for use with saliva samples), their heart rate and blood pressure are taken. They then run on a treadmill and without fluid intake. Over regular intervals of time, their heart rate and blood pressure are taken, their urine output is measured, their saliva is collected and their salivary amylase concentrations recorded and correlated with the increasing dehydration level over time. At the end of the period, the subjects rest, and thereafter, at regular intervals, the subjects are provided with a fixed amount of a fluid of balanced salt, their heart rate and blood pressure are taken, their urine output measured, their saliva is collected and their salivary amylase concentrations recorded and correlated with increasing hydration level over time. For example, the time-course changes in salivary amylase activity from each subject are plotted.

The salivary amylase assay that can be used for this example is as follows: determine the salivary amylase activity (expressed in kU/I) at 37° C. using the enzymatic reagent method with 2-chloro-4-nitrophenyl-4-O-β-D-galactopyranosylmaltoside (Ga1-G2-CNP) as a substrate (Espa AMY liquid2, Nipro Co., Japan) and a clinical automatic analyzer (Miracle Ace 919, Nipro Co., Japan). When Ga1-G2-CNP is hydrolyzed by amylase, the hydrolyzed product (CNP) develops a yellow color with time in the following reaction equation: 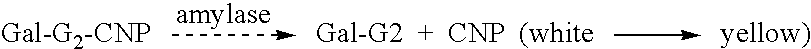
The above enzymatic reaction continues until the substrate is completely consumed. For further details, see M. Yamaguchi et al., Hand-held monitor of sympathetic nervous system using salivary amylase activity and its validation by driver fatigue assessment, Biosens. Biolectron. 21(7): 1007-14 (2006).

Note: Patients who are in shock or who are dehydrated, have increased heart rate and reduced blood pressure. Therefore, this Example also monitors heart rate and blood pressure as additional confirmation of the salivary amylase concentration's correlation with dehydration and shock. The test protocol may be expanded to include a bigger population, according to acceptable statistical analysis. The test protocol may also be applied using test subjects having different parameters, such as: age (e.g., adults, children, seniors, or according to age groups), sex, diet, geographical locations, and any other parameters which may affect salivary amylase concentration or activity, to establish baselines for a population of specific parameter(s).

### Example 5

### Salivary Amylase Activity and Hydration State of Humans Materials and Methods

Three healthy human subjects ages 33, 31, 36 (Subject #1, #2, #3, respectively, shown in Table 1, below) took unstimulated saliva samples of at least 0.5 ml in a state of baseline hydration (Test 1, shown in Table 1, below), then exercised for one hour without consuming any fluid and gave another unstimulated saliva sample of at least 0.5 ml (Test 2, shown in Table 1, below). The subjects then consumed at least one liter of water and thirst was quenched then they produced another saliva sample five minutes after drinking the water: This was also unstimulated and at least 0.5 ml (Test 3, shown in Table 1, below). The samples were immediately frozen to −70 degree C. on dry ice and maintained in such a state until processed. For each Test, two saliva samples were taken from the about 0.5 ml sample above, and tested. Table 1, below, shows the “Values” (spectrophotometrically measured at 405 nm) for the two samples for each Test, the “Mean Value” of the two samples, the mean amylase “Activity” (U/ml of alpha-amylase activity) of the two samples, and the “SD” (“standard deviation”) and “CV” (“coefficient of variability”) for the two samples. Subjects #1 and #2 were tested one day to provide their first sets of data; and then tested another day to provide their second sets of data.

“Unstimulated saliva sample” means that the subjects produced the saliva samples without the aid of artificial external stimulus, such as, by chewing or tasting acidic matter.

The amylase activity levels in the saliva samples were measured using the Salimetrics assay (the salivary alpha-amylase assay kit, commercially available for research use, from Salimetrics LLC, State College, Pa., USA). The testing was performed by Cambridge Biomedical Research Group (Cambridge, Mass., USA) according to the instructions in the kit.

The Salimetrics assay kit used maltotriose with a para-nitrophenol color indicator to measure amylase function. The assay utilized a chromogenic substrate, 2-chloro-p-nitrophenol linked with maltotriose. Wallenfels, K., et al., The enzymic synthesis, by transglucosylation of a homologous series of glycosidically substituted malto-oligosaccharides, and their use as amylase substrates, Carbohydrate Res., 61: 359-368 (1978). The enzymatic action of alpha-amylase on this substrate yielded 2-chloro-p-nitrophenol, which could be spectrophotometrically measured at 405 nm. The amount of alpha-amylase activity present in the sample was directly proportional to the increase in absorbance at 405 nm. For ease of use, the reaction was read in a 96-well microtiter plate with controls provided.

The optical density at 405 nm was measured one minute and then three minutes after combining saliva and the substrate. The activity of amylase was calculated as a function of the amount of substrate digested in that time. The U/ml of alpha-amylase activity in the sample was calculated according to the formula disclosed in the insert of the Salimetrics assay kit.

Discussion

Table 1 shows that with few exceptions, the amylase concentration (as reflected by the amylase activity in Table 1) increased with increased dehydration (comparing Test 1 and Test 2 of Table 1). When the dehydrated subject was subsequently hydrated, the amylase concentration decreased with hydration (comparing Test 2 and Test 3 of Table 1). Without wishing to be bound by the following hypotheses, it is hypothesized that based on the data of Table 1, dehydration reduced salivary flow rate (or the production of saliva), whereas the secretion of amylase remained constant.

It is further hypothesized that: when a person becomes dehydrated the amylase concentration in the saliva increases. This is caused by a relative decrease in the salivary flow rate while the rate of amylase secretion is constant. Upon rehydration the amylase concentration returns to baseline because the saliva flow rate increases back to baseline. It appears that there is no evidence for amylase concentration decreasing with “over-hydration”. Further, saliva amylase secretion is constant in a healthy subject.

TABLE 1

| | | Sample | Mean | | | |
|---|---|---|---|---|---|---|
| Subject | Test | Value | Value | Activity | SD | CV |
| Subject #1 | Test 1 | 176.379 | 167.76 | 110.051 | 12.189 | 7.3 |
| | | 159.141 | | | | |
| | Test 2 | 428.575 | 440.077 | 288.69 | 16.266 | 3.7** |
| | | 451.578 | | | | |
| | Test 3 | 216.723 | 213.684 | 140.176 | 4.298 | 2 |
| | | 210.644 | | | | |
| Subject #2 | Test 1 | 173.358 | 170.494 | 111.844 | 4.051 | 2.4 |
| | | 167.629 | | | | |
| | Test 2 | 349 | 265.267 | 174.015 | 118.417 | 44.6** |
| | | 181.534 | | | | |
| | Test 3 | 387.235 | 380.103 | 249.347 | 10.087 | 2.7 |
| | | 372.97 | | | | |
| Subject #1 | Test 1 | 61.44 | 64.729 | 42.462 | 4.652 | 7.2 |
| Tested on | | 68.019 | | | | |
| a separate | Test 2 | 362.243 | 223.037 | 146.312 | 196.867 | 88.3** |
| day | | 83.832 | | | | |
| | Test 3 | 101.418 | 95.862 | 62.905 | 7.815 | 8.1 |
| | | 90.366 | | | | |
| Subject #2 | Test 1 | 98.519 | 101.118 | 66.334 | 3.676 | 3.6 |
| Tested on | | 103.718 | | | | |
| a separate | Test 2 | 299.39 | 294.21 | 193.002 | 7.327 | 2.5 |
| day | | 289.029 | | | | |
| | Test 3 | 104.153 | 134.657 | 88.335 | 43.139 | 32 |
| | | 165.161 | | | | |
| Subject #3 | Test 1 | 261.11 | 271.921 | 178.38 | 15.29 | 5.6 |
| | | 282.733 | | | | |
| | Test 2 | 378.969 | 390.996 | 256.494 | 17.009 | 4.4 |
| | | 403.024 | | | | |
| | Test 3 | 135.887 | 136.017 | 89.227 | 0.185 | 0.1 |
| | | 136.148 | | | | |

Although the foregoing invention has been described in some detail by way of illustration and example for purposes of clarity and understanding, it will be obvious that various modifications and changes that are within the skill of those skilled in the art are considered to fall within the scope of the appended claims. Future technological advancements that allow for obvious changes in the basic invention herein are also within the claims.

I claim:

1. A method for detecting and quantifying shock or dehydration in a test animal that produces salivary amylase, comprising the steps of:
(a) collecting the saliva of the test animal;
(b) testing for salivary amylase concentration in the collected saliva;
(c) comparing the test salivary amylase concentration with several baselines of salivary amylase concentrations to determine the extent of hydration of the test animal, each baseline represents a certain level of hydration, thus the baselines include a normal baseline representing normal level of hydration, at least one or more dehydration baselines representing different levels of dehydration; and
(d) determining the animal is in a state of shock when the test salivary amylase concentration is below that of the normal level of hydration, and assessing the severity of the shock by matching the test salivary amylase concentrations to the one or more dehydration baselines.

2. The method of claim 1, wherein the different baselines of salivary amylase concentrations are established: (a) for a healthy animal of the same species that is hydrated to provide the normal baseline, and that is dehydrated at one or more different salivary amylase concentrations to provide the respective one or more dehydration baselines; and (b) for an animal of the same species that is in varying degrees of shock.

3. The method of claim 1, further comprising the step of administering treatment to combat dehydration or shock, to the animal, when the animal is determined to be in a state of dehydration or shock.

4. The method of claim 3, wherein the treatment comprises one or more of the following:
intake of fluid or infusions of a balanced salt solution, blood transfusion, and drug administrations.

5. The method of claim 1, wherein the animal is a human.

6. The method of claim 1, wherein the method is used to monitor the progress of shock when the test animal is in shock, wherein the collecting, testing, comparing and determining steps are carried out at different time intervals, and results for the different time intervals are compared to monitor the progress of shock in the test animal.

7. The method of claim 6, further comprising the step of administering one or more anti-shock treatments to the test animal and monitoring the progress of shock to determine the efficacy of the treatments and/or the health progress of the test animal, wherein the anti-shock treatment comprises one or more of the following: infusions of a balanced salt solution, blood transfusion, and drug administrations.

8. The method of claim 1, wherein the salivary amylase assay is based on a system selected from the group consisting of: a biosensor system, a system employing antibodies, and a chemical reaction system.

9. A test kit for determining whether a test subject, who produces salivary amylase, is dehydrated or in shock or the degree of such dehydration or state of shock, said kit comprising:
(a) An assay for measuring the concentration or activity of salivary amylase in a saliva sample of an animal; and
(b) a means for correlating the salivary amylase concentration or activity with hydration level or state of shock of the animal.

10. The test kit of claim 9, wherein said assay and means are contained in a device for a rapid, colorimetric assay for the detection and/or monitoring of the hydration status and/or shock status of an animal that produces salivary amylase, by testing the saliva sample of the animal, comprising:
(a) a test strip comprising a series of spaced-apart reaction zones; and
(b) the reaction zones comprising: (i) a substrate of the salivary amylase, (ii) reagents providing multiple biochemical pathways for the salivary amylase in the saliva sample to convert its substrate to glucose or maltose, and (iii) reagents for the colorimetric detection of the resulting glucose or maltose, to provide visually detectable readout in the presence of the glucose or maltose in order to provide visual determination of the concentration of salivary amylase in the saliva sample or the hydration level of and/or degree of shock suffered by the animal.

11. The test kit of claim 9, wherein said assay and means are contained in a device for rapid, colorimetric assay for the detection and/or monitoring of the hydration and/or shock level of an animal that produces salivary amylase, by testing the saliva sample of the animal, comprising:
(a) a test strip comprising a series of spaced-apart reaction zones; and
(c) the reaction zones comprising: (i) monoclonal antibodies capable of binding salivary amylase in the saliva sample, and (ii) reagents for the colorimetric detection of the resulting monoclonal antibodies bound salivary amylase, to provide visually detectable readout in order to provide visual determination of the concentration of salivary amylase in the saliva sample or the hydration and/or shock level of the animal.

12. A device for assaying the quantity of an analyte in a sample, said device comprising a plurality of reaction zones containing reagents which react with the analyte, wherein: (a) the reaction zones are in different shapes, each shape corresponds to a specified concentration of the analyte that would react with the reagents in the reaction zone; or (b) each reaction zone is associated with a number on the device, each number corresponds to a specified concentration of the analyte that would react with the reagents in the reaction zone.

13. The method of claim 8, wherein the chemical reaction system is selected from the group consisting of: an enzymatic system, a calorimetric system, and a chromogenic system.

14. The use of the test kit of claim 9, to determine as to one or more of the following:
(a) whether the test subject is dehydrated;
(b) whether the test subject in shock; and
(c) the degree the test subject is dehydrated and/or in shock.
